(19) United States
(12) Patent Application Publication
(10) Pub. No.: US 2024/0210321 A1
(43) Pub. Date: Jun. 27, 2024

(54) A SMART TISSUE CLASSIFICATION FRAMEWORK BASED ON MULTI-CLASSIFIER SYSTEMS

(71) Applicant: CytoVeris Inc., Farmington, CT (US)

(72) Inventors: Rishikesh Pandey, Unionville, CT (US); Alan Kersey, South Glastonbury, CT (US); Gary Root, Cheshire, CT (US); Aditya Shirvalkar, Rocky Hill, CT (US)

(21) Appl. No.: 18/567,981

(22) PCT Filed: Jun. 7, 2022
(86) PCT No.: PCT/US2022/032516

§ 371 Date: Dec. 7, 2023

Related U.S. Application Data
(63) Continuation-in-part of application No. PCT/US22/11343, filed on Jan. 5, 2022.
(60) Provisional application No. 63/197,655, filed on Jun. 7, 2021.

(51) Int. Cl. G01N 21/64

(52) U.S. Cl. CPC G01N 21/6486; G01N 21/645; G01N 2021/6417; G01N 2201/1296

(57) ABSTRACT

A method and system of analyzing an ex-vivo tissue sample is provided. The method includes interrogating the tissue sample a plurality of times, each interrogation using at least one excitation light centered on a wavelength distinct from the others, at least one excitation light produces AF emissions from one or more biomolecules associated with the tissue sample, and another is produces diffuse reflectance signals from the tissue sample; b) using a photodetector to detect the AF emissions or diffuse reflectance signals from the tissue sample, producing photodetector signals representative thereof; c) processing the photodetector signals attributable to the AF emissions using a first trained classifier to determine first data sets indicative of biomolecules; d) processing the photodetector signals attributable to the diffuse reflectance signals using a second trained classifier to determine one or more second data sets; and e) determining a type of the tissue sample.

This application claims priority to U.S. Patent Appln. No. 63/197,655 filed Jun. 7, 2021, and PCT Patent Application No. PCT/US2022/011343 filed Jan. 5, 2022, both of which are hereby incorporated by reference in their entirety.

### BACKGROUND OF THE INVENTION

### 1. Technical Area

The present disclosure relates to systems and methods for analyzing excised/ex-vivo tissue samples in general, and to systems and methods for analyzing tissue samples that utilize digital images in particular.

### 2. Background Information

Histopathology remains the gold standard for tissue analysis and identification of cancer. In surgical pathology, after the freshly excised tissue is blocked, it is sent for routine histopathology workflow that involves formalin-fixation, paraffin-embedding (FFPE), microtoming, and staining with various dyes such as hematoxylin and eosin (H&E). The slides are examined by a pathologist under a microscope, and the pathologist's interpretations of the tissue result in the pathology “read” of the sample. However, the entire FFPE process takes days to a week and is labor-intensive and subjective. Advanced optical and electromagnetic (“EM”) imaging approaches have been reported for the determination of tumor margin: These include the use of fluorescence imaging [1-2], optical tomography, radiofrequency spectroscopy, near infrared spectroscopy [3], Raman Spectroscopy [4, 5], and terahertz reflectivity [6].

Among the optical techniques, fluorescence offers a straightforward approach to providing diagnostic information which is interpretable and attributable to known biology. More recently, fluorescence-guided surgery (FGS) has been used for the detection of cancer during surgery and margin assessment [7]. Cancer imaging using FGS typically involves the use of non-specific or targeted fluorescent imaging agents/tracers such as those that bind to cell surface carbohydrates, free proteins, specific enzymes, or expressed cell surface receptors of cancer cells. However, the clinical adaptation of FGS has been hindered due to limited photostability, concern over chemical toxicity, poor tumor to background ratio, and the need for administration of a tracer before surgery.

The biomolecules present in different tissues provide discernible and repeatable autofluorescence [8-11] and reflectance [12] spectral patterns. Intrinsic fluorescence imaging has been used with varying degrees of success in assessing margins. The endogenous fluorescence signatures offer useful information that can be mapped to the functional, metabolic and morphological attributes of a biological sample, and have therefore been utilized for diagnostics purposes. The autofluorescence-based label-free approach offer significant advantages to patients by avoiding potential toxicological issues, FDA approval of contrast agents, the cost of contrast agents, and increased surgical time associated with administering fluorescence imaging agents. The advent of ultraviolet (“UV”) light-emitting diodes (“LEDs”), advancements in UV filter technology, and the emergence of artificial intelligence (“AI”) and machine learning (“ML”) have facilitated fuller exploitation of the rich optical contrast of biomolecular chromophores embedded in tissues.

### SUMMARY OF THE INVENTION

According to an aspect of the present disclosure, a method of analyzing an ex-vivo tissue sample is provided. The method includes: a) sequentially interrogating the tissue sample a plurality of times, each sequential interrogation using at least one excitation light within a plurality of excitation lights and each said excitation light within the plurality of excitation lights centered on a respective wavelength distinct from the respective centered wavelengths of the other excitation lights, wherein at least one of the excitation light centered wavelengths is configured to produce autofluorescence (AF) emissions from one or more biomolecules associated with the tissue sample, and at least one of the excitation light centered wavelengths is configured to produce diffuse reflectance signals from the tissue sample; b) using at least one photodetector to detect the AF emissions, or the diffuse reflectance signals, or both from the tissue sample, and to produce photodetector signals representative of the detected said AF emissions, or the detected said diffuse reflectance signals, or both; c) processing the photodetector signals attributable to the AF emissions using at least one first classifier trained with a plurality of predetermined AF data sets to determine one or more first data sets indicative of biomolecules present within the tissue sample; d) processing the photodetector signals attributable to the diffuse reflectance signals using at least one second classifier trained with a plurality of predetermined diffuse reflectance signal data sets to determine one or more second data sets; and e) determining a type of the tissue sample using the one or more first data sets and the one or more second data sets.

In any of the aspects or embodiments described above and herein, the photodetector signals attributable to the diffuse reflectance signals may provide microstructural information relating to the tissue sample.

In any of the aspects or embodiments described above and herein, the photodetector signals attributable to the diffuse reflectance signals may provide morphological information relating to the tissue sample.

In any of the aspects or embodiments described above and herein, the plurality of predetermined diffuse reflectance signal data sets used to train the second classifier include data sets attributable to known tissue types may include benign tissue, fibrous tissue, adipose tissue, diseased tissue, and tissue morphologies.

In any of the aspects or embodiments described above and herein, the plurality of predetermined AF data sets used to train the first classifier may include data sets attributable to known biomolecules.

In any of the aspects or embodiments described above and herein, the known biomolecules may include at least one of tryptophan, collagen, NADH, FAD, elastin, or hemoglobin.

In any of the aspects or embodiments described above and herein, the at least one first classifier may include a plurality of first classifiers and each producing a first data set, and the step of processing the photodetector signals attributable to the AF emissions may further includes providing the plurality of first data sets to a first metaclassifier, and the step of determining the type of the tissue sample may utilize an output of the first metaclassifier.

In any of the aspects or embodiments described above and herein, the at least one first classifier may include a plurality of first classifiers and each producing a first data set, and the at least one second classifier may include a plurality of second classifiers and each producing a second data set, and the step of processing the photodetector signals attributable to the AF emissions may further include providing the plurality of first data sets to a first metaclassifier, and the step of processing the photodetector signals attributable to the diffuse reflectance signals may include providing the plurality of second data sets to the first metaclassifier, and the step of determining the type of the tissue sample may utilize an output of the first metaclassifier.

In any of the aspects or embodiments described above and herein, the method may further include processing the photodetector signals attributable to the diffuse reflectance signals using at least one third classifier trained with a plurality of predetermined morphology signal data sets to determine one or more third data sets indicative of morphologies present within the tissue sample, and the step of determining the type of the tissue sample may further use the one or more third data sets indicative of morphologies present within the tissue sample.

In any of the aspects or embodiments described above and herein, the step of processing the photodetector signals attributable to the AF emissions using at least one first classifier may further include providing the plurality of first data sets to a first metaclassifier, the step of processing the photodetector signals attributable to the diffuse reflectance signals using at least one second classifier may include providing the plurality of second data sets to the first metaclassifier, and the step of processing the photodetector signals attributable to the diffuse reflectance signals using at least one third classifier trained with a plurality of predetermined morphology signal data sets may include providing the plurality of third data sets to the first metaclassifier, and the step of determining the type of the tissue sample may utilize an output of the first metaclassifier.

In any of the aspects or embodiments described above and herein, the step of determining the type of the tissue sample may utilize a third classifier that utilizes the one or more first data sets from the at least one first classifier and the one or more second data sets from the at least one second classifier in a cascading manner.

In any of the aspects or embodiments described above and herein, the step of processing the photodetector signals attributable to the AF emissions and the step of processing the photodetector signals attributable to the diffuse reflectance signals may further include providing a first output from the at least one first classifier and a second output from the at least one second classifier to a second level classifier in an ensemble classifier architecture, and the step of determining the type of the tissue sample may utilize a third output from the second level classifier.

According to another aspect of the present disclosure, a system for analyzing an ex-vivo tissue sample is provided that includes an excitation light source, at least one photodetector, and a system controller. The excitation light source is configured to selectively produce a plurality of excitation lights. Each excitation light is centered on a wavelength distinct from the centered wavelength of the other said excitation lights. At least one of the excitation light centered wavelengths is configured to produce AF emissions from one or more biomolecules associated with a bladder wall tissue, and diffuse reflectance signals from the tissue sample. The system is configured so that the plurality of excitation lights are incident to the tissue sample. The at least one photodetector is configured to detect the AF emissions, or the diffuse reflectance signals, or both from the tissue sample as a result of the respective incident excitation light, and to produce signals representative of the detected AF emissions, or the detected diffuse reflectance signals, or both. The system controller is in communication with the excitation light source, the at least one photodetector, and a non-transitory memory storing instructions, which instructions when executed cause the system controller to: a) control the excitation light unit to sequentially produce the plurality of excitation lights; b) control the at least one photodetector to detect the AF emissions, or the diffuse reflectance signals, or both from the tissue sample, and to produce photodetector signals representative of the detected said AF emissions, or the detected said diffuse reflectance signals, or both; c) process the photodetector signals attributable to the AF emissions using at least one first classifier trained with a plurality of predetermined AF data sets to determine one or more first data sets indicative of biomolecules present within the tissue sample; d) process the photodetector signals attributable to the diffuse reflectance signals using at least one second classifier trained with a plurality of predetermined diffuse reflectance signal data sets to determine one or more second data sets; and e) determine a type of the tissue sample using the one or more first data sets and the one or more second data sets.

In any of the aspects or embodiments described above and herein, the at least one first classifier may include a plurality of first classifiers, each producing a first data set. The instructions that when executed cause the system controller to process the photodetector signals attributable to the AF emissions, may further cause the system controller to provide the plurality of first data sets to a first metaclassifier, and the determination of the tissue sample type may utilize an output of the first metaclassifier.

In any of the aspects or embodiments described above and herein, the at least one first classifier may include a plurality of first classifiers, each producing a first data set. The at least one second classifier may include a plurality of second classifiers, each producing a second data set. The instructions that when executed cause the system controller to process the photodetector signals attributable to the AF emissions, may further cause the system controller to provide the plurality of first data sets to a first metaclassifier. The instructions that when executed cause the system controller to process the photodetector signals attributable to the diffuse reflectance signals, may further cause the system controller to provide the plurality of second data sets to the first metaclassifier. The instructions that when executed cause the system controller to determine the type of the tissue sample may utilize an output of the first metaclassifier.

In any of the aspects or embodiments described above and herein, the instructions that when executed may further cause the system controller to process the photodetector signals attributable to the diffuse reflectance signals using at least one third classifier trained with a plurality of predetermined morphology signal data sets to determine one or more third data sets indicative of morphologies present within the tissue sample. The instructions that when executed cause the system controller to determine the type of the tissue sample may further use the one or more third data sets indicative of morphologies present within the tissue sample.

In any of the aspects or embodiments described above and herein, the instructions that when executed cause the system controller to process the photodetector signals attributable to the AF emissions using the at least one first classifier may further cause the system controller to provide the plurality of first data sets to a first metaclassifier. The instructions that when executed cause the system controller to process the photodetector signals attributable to the diffuse reflectance signals using the at least one second classifier may further cause the system controller to provide the plurality of second data sets to the first metaclassifier. The instructions that when executed cause the system controller to process the photodetector signals attributable to the diffuse reflectance signals using the at least one third classifier trained with a plurality of predetermined morphology signal data sets may further cause the system controller to provide the plurality of third data sets to the first metaclassifier. The instructions that when executed cause the system controller to determine the type of the tissue sample may use an output of the first metaclassifier.

In any of the aspects or embodiments described above and herein, the instructions that when executed cause the system controller to determine the type of the tissue sample may utilize a third classifier that utilizes the one or more first data sets from the at least one first classifier and the one or more second data sets from the at least one second classifier in a cascading manner.

In any of the aspects or embodiments described above and herein, the instructions that when executed cause the system controller to process the photodetector signals attributable to the AF emissions and to process the photodetector signals attributable to the diffuse reflectance signals may further cause the system to provide a first output from the at least one first classifier and a second output from the at least one second classifier to a second level classifier in an ensemble classifier architecture. The instructions that when executed cause the system controller to determine the type of the tissue sample may utilize a third output from the second level classifier.

The foregoing features and elements may be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIG. 1 is a diagrammatic illustration of a present disclosure system embodiment.

FIG. 2 is a table of excitation/illumination wavelengths versus reflectance/fluorescence wavelengths.

FIG. 3 is a graph of fluorescence intensity versus fluorescence emission wavelength, illustrating diagrammatic representations of biomolecule curves.

FIG. 4 is a schematic representation of a single classification model.

FIG. 5 is a schematic representation of an ensemble classification model.

FIG. 6A is a schematic representation of an ensemble classifier architecture that includes a plurality of classifiers trained on different AF data sets providing input to a metaclassifier.

FIG. 6B is a schematic representation of an ensemble classifier architecture that includes a plurality of classifiers trained on different AF and reflectance data sets providing input to a metaclassifier.

FIG. 7 is a schematic representation of an ensemble classifier architecture that uses a plurality of classifiers trained on multispectral AF data sets and tissue morphology data sets providing input to a metaclassifier.

FIG. 8A is a schematic representation of a cascading classification architecture.

FIG. 8B is a schematic representation of a cascading classification architecture.

FIG. 9 is a schematic representation of a multi-stage ensemble classifier architecture that includes a classifier trained to determine adipose tissue providing input to an ensemble classifier.

FIG. 10 is a schematic representation of a multi-stage ensemble classifier architecture that includes a classifier trained to determine adipose tissue providing input to an ensemble classifier.

FIG. 11 is a schematic representation of a multi-stage classification architecture.

### DETAILED DISCLOSURE

The present disclosure is directed to a novel dye-free multimodal optical approach that combines multispectral autofluorescence (“AF”) imaging with multispectral reflectance imaging to measure both tissue emission and absorption characteristics to provide comprehensive analysis and profiling of excised/ex-vivo tissue. The present disclosure system includes an excitation light source, one or more photodetectors, a system controller, as well as other components. As will be described herein, embodiments of the present disclosure are configured for imaging/analysis of ex-vivo tissue samples.

Biomolecules present in different tissues provide discernible and repeatable AF and reflectance spectral patterns. The endogenous fluorescence signatures offer useful information that can be mapped to functional, metabolic, and/or morphological attributes of a biological sample, and therefore may be used for diagnostic purposes. Biomolecular changes occurring in the cell and tissue state during pathological processes and disease progression result in alterations of the amount and distribution of endogenous fluorophores and can form the basis for tissue/cancer identification. Tissue AF has been proposed to detect various malignancies including cancer by measuring either differential intensity or lifetimes of the intrinsic fluorophores. Biomolecular constituents such as tryptophan, collagen, elastin, nicotinamide adenine dinucleotide (NADH), flavin adenine dinucleotide (FAD), elastin, porphyrins, etc. present in tissue provide discernible and repeatable autofluorescence spectral patterns.

The excitation light source may include one or more excitation light units. In some embodiments, an excitation light unit may be configured to produce excitation light centered at a particular wavelength. In those system embodiments that include a plurality of excitation light units, different excitation light units may be configured to produce excitation light centered at different wavelengths; e.g., a first excitation light unit configured to produce excitation light centered at wavelength “X”, a second excitation light unit configured to produce excitation light centered at wavelength “Y”, and the like. In some embodiments, the excitation light source may be or include a white light source. For example, the system may include a white light source in combination with one or more filters that collectively produce excitation light centered at different wavelengths. In some embodiments, the system may include a white light source used to interrogate the sample unfiltered; e.g., for registration purposes, or the like.

An excitation light unit may be configured to produce AF emissions from a tissue sample and/or may be configured to produce reflectance signals from a tissue sample. Non-limiting examples of acceptable excitation light sources include lasers and light emitting diodes (LEDs) that may be centered at particular wavelengths, or a tunable excitation light source configured to selectively produce light centered at respective different wavelengths. An example of an acceptable white light source is a flash lamp. The present disclosure is not limited to any particular type of excitation light unit. In those embodiments wherein an excitation light unit is configured to produce light centered on a particular wavelength, the respective wavelength may be chosen based on the photometric properties associated with one or more biomolecules (or tissue type, etc.) of interest. Excitation light incident to a biomolecule that acts as a fluorophore will cause the fluorophore to emit fluorescent light at a wavelength longer than the wavelength of the excitation light; i.e., via AF.

As stated above, tissue may naturally include certain fluorophores such as tryptophan, collagen, elastin, nicotinamide adenine dinucleotide (NADH), flavin adenine dinucleotide (FAD), elastin, porphyrins, and the like. In addition, biomolecular changes occurring in the cell and tissue state during pathological processes and as a result of disease progression often result in alterations of the amount and distribution of these endogenous fluorophores. Hence, different tissue types and states can exhibit distinct intrinsic tissue AF, or in other words an “AF signature”, that is readily identifiable. Embodiments of the present disclosure may utilize these AF characteristics/signatures to identify different tissue types and/or tissue constituents.

Excitation wavelengths may also be chosen that cause detectable light reflectance from tissue of interest. The detectable light reflectance is a function of light absorption of the tissue and/or light scattering associated with the tissue (this may be collectively referred to as diffuse reflectance). Certain tissue types or permutations thereof, or constituents thereof, have differing and detectable light reflectance characteristics (“signatures”) at certain wavelengths. Significantly, these reflectance characteristics can provide information beyond intensity; e.g., information relating to cellular or microcellular structure such as cell nucleus and extracellular components. The morphology of a first type healthy tissue cell may be different from that of a second type healthy cell, and/or different from an abnormal or diseased tissue cell. Hence, the ability to gather cellular or microstructural morphological information (sometimes referred to as “texture”) provides another tool for determining tissue types and the state and characteristics of such tissue.

The excitation light source may be configured to produce light at wavelengths in the ultraviolet (UV) region (e.g., about 100-400 nm) and in some applications may include light in the visible region (e.g., 400-700 nm). The excitation light wavelengths may be chosen based on the photometric characteristics of the biomolecules of interest (e.g., AF and absorption) and the present disclosure is not, therefore, limited to the exemplary wavelength ranges disclosed above.

Regarding the one or more photodetectors within the system, the present disclosure may utilize a variety of different photodetector types configured to sense light and provide signals that may be used to measure the same. Non-limiting examples of an acceptable photodetector include those that convert light energy into an electrical signal such as photodiodes, avalanche photodiodes, a charge coupled device (“CCD”) array, an intensified charge coupled device (“ICCD”) array, a complementary metal-oxide-semiconductor (“CMOS”) image sensor, or the like. The photodetector may take the form of a camera. As will be described below, the one or more photodetector(s) are configured to detect AF emissions from the interrogated tissue and/or diffuse reflectance from the interrogated tissue and produce signals representative of the detected light and communicate the signals to the system controller.

The system controller is in communication with other system components such as the light source and the light detector and may be in communication with other system components. The system controller may be in communication with system components to control the operation of the respective component and/or to receive signals from and/or transmit signals to that component to perform the functions described herein. The system controller may include any type of computing device, computational circuit, processor(s), CPU, computer, or the like capable of executing a series of instructions that are stored in memory. The instructions may include an operating system, and/or executable software modules such as program files, system data, buffers, drivers, utilities, and the like. The executable instructions may apply to any functionality described herein to enable the system to accomplish the same algorithmically and/or coordination of system components. The system controller includes or is in communication with one or more memory devices. The present disclosure is not limited to any particular type of memory device, and the memory device may store instructions and/or data in a non-transitory manner. Examples of memory devices that may be used include read-only memory, random access memory, volatile memory, non-volatile memory, static memory, dynamic memory, flash memory, cache memory, and/or any device that stores digital information. The system controller may include, or may be in communication with, an input device that enables a user to enter data and/or instructions, and may include, or be in communication with, an output device configured, for example to display information (e.g., a visual display or a printer), or to transfer data, etc. Communications between the system controller and other system components may be via a hardwire connection or via a wireless connection.

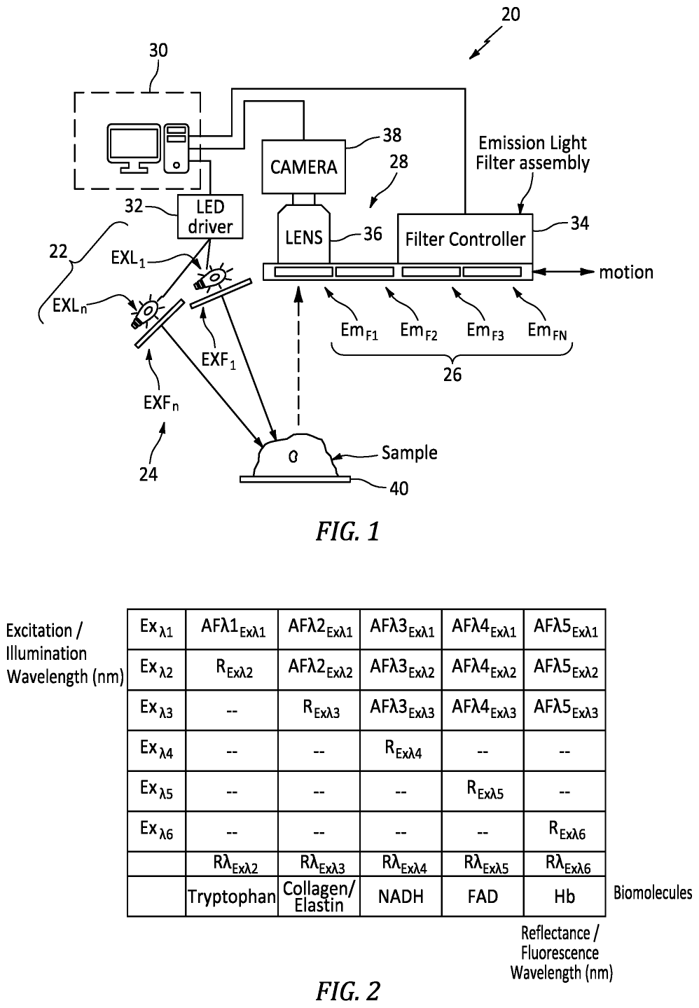

Some embodiments of the present disclosure may include optical filtering elements configured to filter excitation light, or optical filtering elements configured to filter emitted light (including reflected light), or both. Each optical filtering element may be configured to pass a defined bandpass of wavelengths associated with an excitation light source or emitted/reflected light (e.g., fluorescence or reflectance), and may take the form of a bandpass filter. Regarding filtering excitation light, the system may include an independent filtering element associated with each independent excitation light source or may include a plurality of filtering elements disposed in a movable form (e.g., a wheel or a linear array configuration) or may include a single filtering element that is operable to filter excitation light at a plurality of different wavelengths, or each excitation light source may be configured to include a filtering element, or the like. Regarding filtering emitted or reflected light, the system may include a plurality of independent filtering elements each associated with a different bandwidth or may include a plurality of filtering elements disposed in a movable form or may include a single filtering element that is operable to filter emitted/reflected light at a plurality of different wavelengths, or the like. The bandwidth of the emitted/reflected light filters are typically chosen based on the photometric properties associated with one or more biomolecules of interest. Certain biomolecules may have multiple emission or reflectance peaks. The bandwidth of the emitted/reflected light filters are typically chosen to allow only emitted/reflected light from a limited portion of the biomolecule emission/reflectance response; i.e., a portion of interest that facilitates the analysis described herein. As will be described below, the exemplary system embodiment shown in FIG. 1 illustrates a non-limiting example of optical filtering. In some embodiments, the system may include a tunable bandpass filter that is controllable to provide a plurality of different bandwidth filtration modes. In certain embodiments, the excitation filter may be disposed or integrated as a part of excitation light source. For example, the LED or other light source can be coated with a material to allow desired bandpass.

An exemplary embodiment of a present disclosure system 20 is diagrammatically illustrated in FIG. 1. This system 20 embodiment includes an excitation light source 22, an excitation light filter arrangement 24, an emission/reflectance light filter assembly 26, a photodetector arrangement 28, and a system controller 30. The excitation light source 22 includes a plurality of independent excitation light sources (e.g., EXL₁ . . . EXLₙ, where “n” is an integer greater than one), each operable to produce an excitation light centered at a particular wavelength and each centered on an excitation wavelength different from the others. The independent excitation light sources are directly or indirectly in communication with the system controller 30. In this example, the independent excitation light sources are UV LEDs. As described above, the wavelengths produced by the independent excitation light sources are chosen based on the photometric properties associated with biomolecules/tissue types of interest. The LEDs are in communication with an LED driver 32 that may be independent of the system controller 30 or the functionality of the LED driver 32 may be incorporated into the system controller 30. The excitation light filter arrangement 24 shown in FIG. 1 includes an independent bandpass filter (EXF₁ . . . EXFₙ) for each excitation light source and the bandwidth filter properties for each independent bandpass filter are tailored for the respective excitation light source with which it is associated. In alternative embodiments the system 20 may be configured without an excitation light filter arrangement 24, or each excitation light source may have an incorporated filter element, or the system 20 may include an excitation light filter arrangement 24 with a movable filter element (e.g., a wheel, linear array, etc.), or may include a single filtering element that is operable to filter excitation light at a plurality of different wavelengths. The system 20 embodiment diagrammatically shown in FIG. 1 includes an emission light filter assembly 26 having a filter controller 34 and a linear array of bandpass filters (e.g., Em_{F1}, Em_{F2} . . . Em_{FN}). The filter controller 34 is configured to selectively position each respective bandpass filter in a light path between the tissue sample (i.e., the source of the emitted/reflected light) and the photodetector arrangement 28 to permit filtering of the emitted/reflected light prior to detection by the photodetector arrangement 28. The filter controller 34 may be in communication with the system controller 30, or the filter controller 34 functionality may be incorporated into the system controller 30. As stated above, the bandwidth of the respective bandpass filters for the emitted/reflected light are typically chosen based on the photometric properties associated with one or more biomolecules of interest; e.g., to allow only emitted/reflected light from a limited portion of the biomolecule emission/reflectance response that is of interest to facilitate the analyses described herein. In alternative embodiments the system 20 may be configured without an emission light filter assembly 26 or may include an emission light filter assembly 26 configured differently from the configuration diagrammatically shown in FIG. 1. The photodetector arrangement 28 may include a lens arrangement 36 and a camera 38. The lens arrangement 36 is configurable to suit the application at hand. For example, in some embodiments the lens arrangement 36 may include a single fixed focus lens. In some embodiments, the lens arrangement 36 may be configured to address chromatic dispersion. For example, the lens arrangement 36 may include one or more corrective lenses configured to address aberration/focus as may be desired. In some embodiments, the lens arrangement 36 may be controllable to selectively change lens configurations and is in communication with the system controller 30. The camera 38 is configured to produce signals representative of the sensed emitted/reflected light passed through the emission light filter assembly 26. The aforesaid signals may be referred to as an “image” or may be processed into an image. The camera 38 is in communication with the system controller 30.

In the operation of the system 20 embodiment diagrammatically shown in FIG. 1, an excised tissue sample may be placed on a stage 40 or other platform at a position optically aligned with the photodetector arrangement 28. In some instances, the system 20 and/or the tissue sample may be such that the entirety of the sample can be imaged without changing the relative positions of the tissue sample and the system optics. In other instances, wherein the system 20 is not configured to image the entirety of the tissue sample, the system 20 may be configured to move one or both of the tissue sample and the system optics relative to one another so multiple regions of the tissue sample may be imaged; e.g., the tissue sample may be scanned. The images from the respective regions may subsequently be “stitched” together to form one or more images of the entirety of the tissue sample. The system controller 30 (through stored instructions) is configured to sequentially operate the independent excitation light sources (e.g., EXL₁ . . . EXLₙ). As each excitation light source is operated, the produced excitation light may pass through an excitation light filter prior to being incident to the tissue sample. If a fluorophore of interest is present within the tissue sample and that fluorophore is responsive to the wavelength of the incident excitation light, the excitation light will cause the fluorophore to produce an AF emission at a wavelength that is different from the excitation wavelength. Excitation light centered on a particular wavelength may produce AF emissions from more than one fluorophore of interest. Referring to the table in FIG. 2, a first excitation wavelength (EXλ1) may produce AF emissions at several different wavelengths (AFλ1_{EXλ1}, AFλ2_{EXλ1}, AFλ3_{EXλ1}, AFλ4_{EXλ1}, AFλ5_{EXλ1}). The same excitation light incident to the tissue sample may also generate diffuse reflectance signals; i.e., excitation light that is reflected from the tissue sample. For example, and again referring to the table in FIG. 2, a second excitation wavelength (EXλ2) can produce reflectance signals (R_{EXλ2}) and AF emissions at several different wavelengths (AFλ2_{EXλ2}, AFλ3_{EXλ2}, AFλ4_{EXλ2}, AFλ5_{EXλ2}), a third excitation wavelength (EXλ3) can produce reflectance signal (R_{EXλ3}) and AF emissions at several different wavelengths (AFλ3_{EXλ3}, AFλ4_{EXλ3}, AFλ5_{EXλ4},), and so on. The emission/reflectance light filter assembly 26 is controlled to coordinate placement of a particular bandpass filter in alignment with the camera 38, which bandpass filter is appropriate for the excitation light source being operated and to produce a limited bandwidth of the emitted/reflected light that is of interest for the analysis at hand; e.g., associated with particular biomolecules of interest. Some amount of the emitted light passes through the bandpass filter, is sensed by the camera 38, and the camera 38 produces signals representative of the sensed emitted/reflected light. The aforesaid signals may be referred to as an image or may be processed into an image. In some applications, an excitation wavelength may be chosen only for AF emissions of interest (e.g., EXλ1 in FIG. 2), and/or an excitation wavelength may be chosen only for diffuse reflectance signals of interest (e.g., EXλ4, EXλ5, and EXλ6 in FIG. 2). The above described process is repeated until the sample has been examined using all of the desired wavelengths of excitation light. As will be detailed below, the respective images may be used to collectively identify biomolecules/tissue types of interest with a desirable degree of specificity and sensitivity.

In the system embodiment described above and others, the signals (i.e., image) representative of the emitted light (AF and/or reflectance) captured by the photodetector arrangement (e.g., camera or plurality of photodetectors) for each excitation light wavelength may collectively provide a mosaic of information relating to the tissue sample. The chart shown in FIG. 2 illustrates an exemplary scenario wherein six (6) different excitation light sources, each centered on a different wavelength (i.e., Ex_{λ1}, Ex_{λ2}, Ex_{λ3}, Ex_{λ4}, Ex_{λ5}, and Ex_{λ6} nm), are used within the system. Depending on the presence of certain fluorophores within the tissue sample, the first excitation wavelength (i.e., Ex_{λ1}) may produce AF emissions of interest at five (5) different wavelengths (AFλ1_{Exλ1}, AFλ2_{Exλ1}, AFλ3_{Exλ1}, AFλ4_{Exλ1}, AFλ5_{Exλ1}), and the second excitation wavelength (i.e., Ex_{λ2}) may produce AF emissions of interest at four (4) different wavelengths (AFλ2_{Exλ2}, AFλ3_{Exλ2}, AFλ4_{Exλ2}, AFλ5_{Exλ2}), and so on. The second excitation wavelength (i.e., Ex_{λ2}) may also produce a reflectance image at this wavelength (R_{Exλ2}) that is a useful indicator of the presence or absence of certain tissue types within the tissue sample. The Ex_{λ4}, Ex_{λ5}, and Ex_{λ6} excitation wavelengths may not be used to produce AF emissions of interest, but each may be used to produce a reflectance image of interest (i.e., R_{Exλ4}, R_{Exλ5}, R_{Exλ6}). As can be seen from the example shown in FIG. 2, the six (6) excitation wavelengths (i.e., Ex_{λ1}, Ex_{λ2}, Ex_{λ3}, Ex_{λ4}, Ex_{λ5}, and Ex_{λ6} nm) may be used to produce seventeen emitted light images (AFλ1_{Exλ1}, AFλ2_{Exλ1}, AFλ3_{Exλ1}, AFλ4_{Exλ1}, AFλ5_{Exλ1}, R_{Exλ2}, AFλ2_{Exλ2}, AFλ2_{Exλ2}, AFλ4_{Exλ2}, AFλ5_{Exλ2}, R_{Exλ3}, AFλ3_{Exλ3}, AFλ4_{Exλ3}, AFλ5_{Exλ3}, R_{Exλ4}, R_{Exλ5}, R_{Exλ6}) that may be used collectively to identify biomolecule/tissue types of interest with a desirable degree of specificity and sensitivity. It should be noted that the number of excitation wavelengths, the number of reflectance wavelengths, the biomolecule, and the particular AF emissions selected, and reflectance emissions indicated in FIG. 2 are provided to illustrate the present disclosure, and the present disclosure is not limited to this example. For example, the analysis of different types of tissue may benefit from fewer or more excitation wavelengths, different biomolecule, etc.

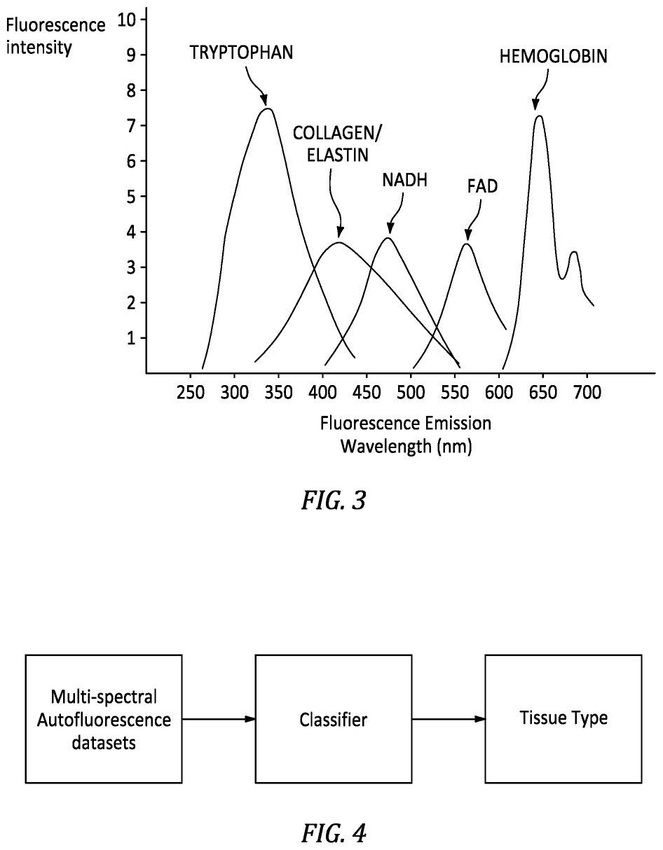

The integrated information provided by the aforesaid emitted light images provide distinct benefits in the process of identifying biomolecule/tissue types of interest with a desirable degree of specificity and sensitivity. As can be seen from FIG. 3, AF emissions are produced in a peaked band with an intensity value that is centered on a particular wavelength. Hence, AF emissions centered on a particular wavelength will include AF emissions not only on the peak wavelength but also on adjacent wavelengths albeit at a lesser intensity. As can also be seen in FIG. 3, the biomolecule/fluorophores of interest (e.g., tryptophan, collagen, NADH, FAD, elastin, hemoglobin, etc.) have characteristic AF intensity curves with a peak centered on a wavelength but also including lesser intensities at wavelengths adjacent the peak wavelength. The AF intensity curves of some of the biomolecules may overlap to a degree. As a result, AF emissions at a particular wavelength within the overlap region may be a product of AF emissions from a first biomolecule or from a second biomolecule and are likely not dispositive by themselves of either biomolecule. As indicated above, at least some biomolecules of interest also have reflectance curves (indicating the amount of light reflectance which is a function of light absorption of the tissue and light scattering within the tissue) with a peak centered on a peak wavelength but also including lesser intensities at wavelengths adjacent the peak wavelength. The reflectance curves of some of the biomolecules may also overlap to a degree. As a result, reflectance at a particular wavelength within the overlap region may be a product of reflectance from a first biomolecule or from a second biomolecule and is likely not dispositive by itself of either biomolecule. In addition, as indicated above, reflectance images can also provide cellular or tissue microstructural information (e.g., morphology) that can be used as an additional tool for determining tissue types and the state of such tissue. The collective information provided by the aforesaid plurality of emitted/reflected light images produced by the present disclosure system, however, provides distinct information at different excitation wavelengths that can be used to identify biomolecule/tissue types with a desirable degree of specificity and sensitivity.

Embodiments of the present disclosure may include a plurality of classifiers using advanced machine learning and/or AI algorithms on multispectral autofluorescence data sets, reflectance data sets, and combinations thereof to fully exploit the biochemical information content (e.g., from fluorescence) and morphological information content (e.g., both reflectance and fluorescence). This rapid and label-free approach, which offers cost-effectiveness and ease-of-use, has the potential to provide significant advantages in surgical and pathological settings.

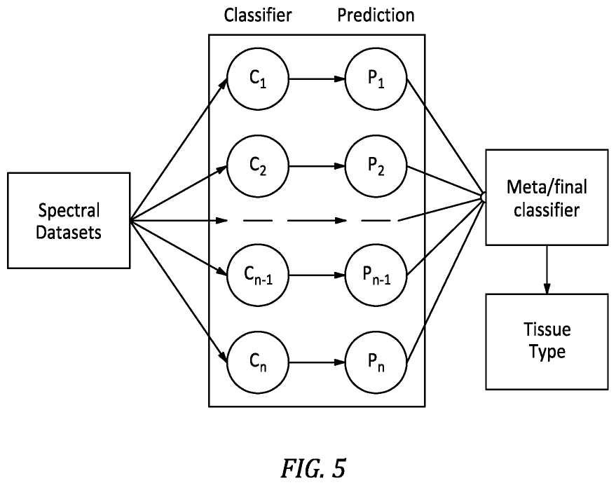

For the AF imaging, either an entire image, or characteristics (e.g., pixel intensity) of an image, or a portion of an image (e.g., a localized grouping of pixels, sometimes referred to as a “superpixel”), or any combination thereof may be used for classification algorithm development. Single classification methods such as logistic regression, discriminant analysis, support vector machine, random forest, XG boost, and the like have been used in the past for the classification task even on hyperspectral images [13] as illustrated in FIG. 4. Ensemble classification models have been used in the past [14, 15], including in some spectroscopic applications. However, these ensemble classification models typically either use different features of the sample encoded from the same optical techniques or use the same data sets produced using the same preprocessing steps; e.g., see FIG. 5. As shown in FIG. 5, classifiers C₁-Cₙ produce predictions P₁-Pₙ.

Aspects of the present disclosure utilize a novel hybrid classification approach that uses both morphological and molecular attributes of the tissues derived from AF images that may be acquired at a plurality of different excitation and emission wavelengths, and from reflectance images obtained using one or more optical techniques. Some embodiments of the present disclosure may combine AF imaging with reflectance imaging to compliment and increase the biomolecular and morphological information content and thereby increase the diagnostic power. Aspects of the present disclosure therefore leverage the fact that different tissue types absorb different amounts of light at specific wavelengths and therefore the combination and autofluorescence and reflectance imaging offers higher diagnostic power.

The stored instructions within the system controller 30 may include a plurality of trained classifiers, each “trained” using a clinically significant number of images of known tissue types (e.g., including but not limited to benign tissue types, fibrous tissue types, adipose/fat tissue types, diseased tissue types (e.g., cancerous), abnormal tissue types, tissue morphologies, etc.) and features collected at the respective excitation wavelengths. Alternatively, the system controller 30 may be in communication with a plurality of trained classifiers. The trained classifiers in turn may be used to evaluate the acquired light images (AF and/or reflectance) collected from the tissue sample at the various different excitation/emission wavelengths to determine the presence or absence of biomolecule/tissue types/features of interest. The present disclosure is not limited to any particular type of classifier; e.g., some classifiers may employ classification methodologies/algorithms such as dictionary learning, anomaly detector, convolutional neural network (CNN), deep neural network (DNN), logical regression, discriminate analysis, support vector machine (SVM), random forest, XG boost, and the like. As will be described below, a classifier may produce a binary output or a probability output, etc. In some embodiments, an ensemble classifier consisting of two or more classifiers based on the same or different classification methods can be utilized. An ensemble classifier may utilize input from one or more base classifiers provided to a second level classifier trained on such input. The second level classifier processes the aforesaid base classifier input (e.g., predictions) to produce an output that typically has a greater probability/accuracy than is produced by the constituent base classifiers. The present disclosure is not limited to these examples.

During the classifier training process, an imaging system (e.g., the same as or similar to the photodetectors described above in FIG. 1—e.g., a camera 38) may be used to acquire a clinically significant number of multispectral images. The “clinically significant number of images” may vary depending on the classifier(s) used and the target tissue type and/or target tissue constituent but will include a number of images adequate to enable the classifier to perform with an acceptable degree of certainty for the task at hand. As stated above, embodiments of the present disclosure may use multispectral AF imaging and multispectral reflectance imaging to measure both tissue absorption and emission/reflectance characteristics. The multispectral AF imaging may include sequentially interrogating the tissue sample with light centered on a “N” number of different wavelengths, where “N” is an integer and is equal to or greater than one. In similar fashion, the multispectral reflectance imaging may include sequentially interrogating the tissue sample with light centered on a “M” number of different wavelengths, where “M” is an integer and is equal to or greater than one. To be clear and as described above, in some instances a tissue type may be identified using only multispectral AF, or only multispectral reflectance, or tissue identification may be accomplished using some combination of multispectral AF and multispectral reflectance. The acquired images may be processed to provide desirable uniformity to the respective images. The processing may include, but is not limited to, altering the images to a uniform size or format, adjusting image intensity to a uniform level, aligning the images, and the like. The processed images may then be input to a respective classifier and a pathological analysis of each image that includes a tissue type determination based on the entirety of the respective image, or a read of segments of respective images or features present within the respective images (e.g., intensity, intensity ratios, texture information, etc.), or the like, may be included.

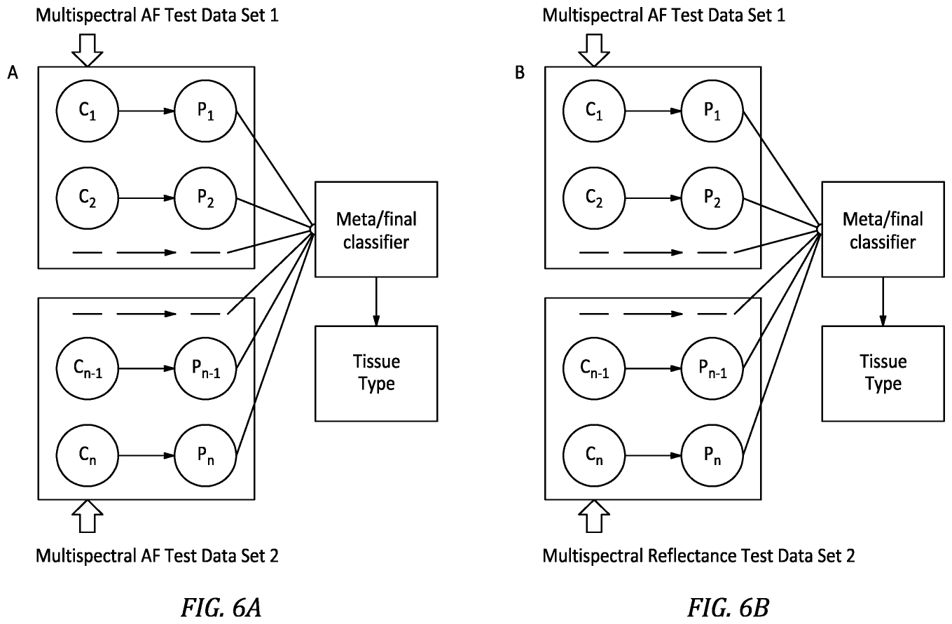

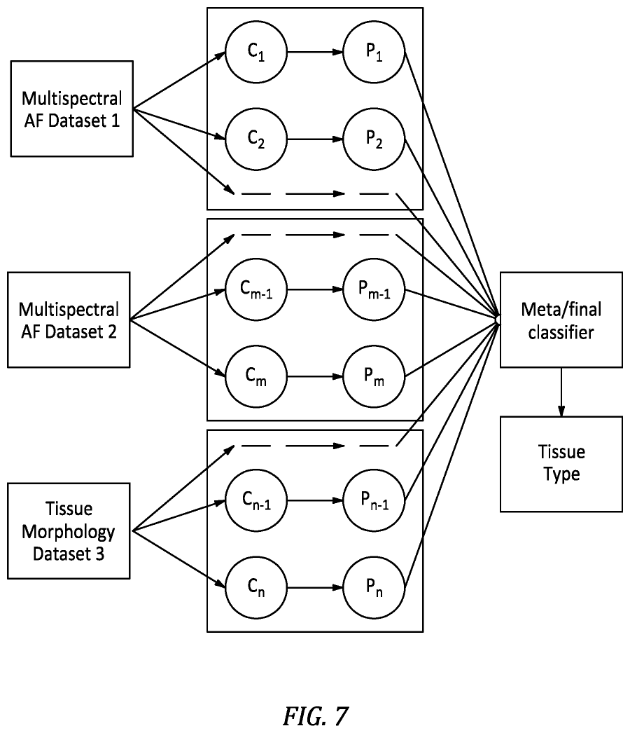

Different embodiments of the present disclosure hybrid classification framework are illustrated in the present figures. FIG. 6A illustrates a classification architecture in which one or more classifiers—trained on different AF data sets may be combined. FIG. 6B illustrates a classification architecture in which one or more classifiers—trained on different AF and reflectance data sets—may be combined. As shown in FIGS. 6A and 6B, classifiers C₁ produces predictions P₁, C₂ produces predictions P₂, Cₙ₋₁ produces predictions Pₙ₋₁, and Cₙ produces predictions Pₙ. In other embodiments of the present disclosure, an ensemble-classifier may be utilized that uses a plurality of multispectral AF data sets with morphological (e.g., tissue textural data) attributes—see FIG. 7. The AF data sets are different as they may consist of different panels of multi-spectral images and may differ in preprocessing steps. The predictions from the base classifiers may then be combined to generate a final classifier. As shown in FIG. 7, classifiers C₁ produces predictions P₁, C₂ produces predictions P₂, Cₘ₋₁ produces predictions Pₘ₋₁, Cₘ produces predictions Pₘ, Cₙ₋₁ produces predictions Pₙ₋₁, and Cₙ produces predictions Pₙ. Non-limiting examples of a combination may include taking an average or weighted average of diagnostic metrics or may be based on a voting system to combine the individual predictions to arrive at final predictions.

In some instances, a meta-classifier may be employed that uses machine learning/AI to combine predictions from individual base classifiers. In some instances, these classifiers may be selectively tuned to capture and maximize different aspects of classification, and their impact on the final stage can be weighted by the machine-learning training data sets. In addition, various features extracted from attribute-learning base classifiers (ABCs) might be used as input to the AI/ML-based meta classifier. Some features may be correlated to different endogenous biomolecules such as tryptophan, collagen, NADH, FAD, and the like to facilitate development of an explainable AI (XAI) or ML classifier.

The present disclosure is not limited to either AI, machine learning, or multivariate models and any combination of these techniques may be used. In some instances, classifiers trained on differing spatial dimensions or image patch size may be combined to account for both local and global tissue morphologies. For example, a classifier trained on a smaller tile will encode a more localized cellular structure, whereas a classifier trained on a bigger tile may account for tissue microenvironment of neighborhood and cancer field effect.

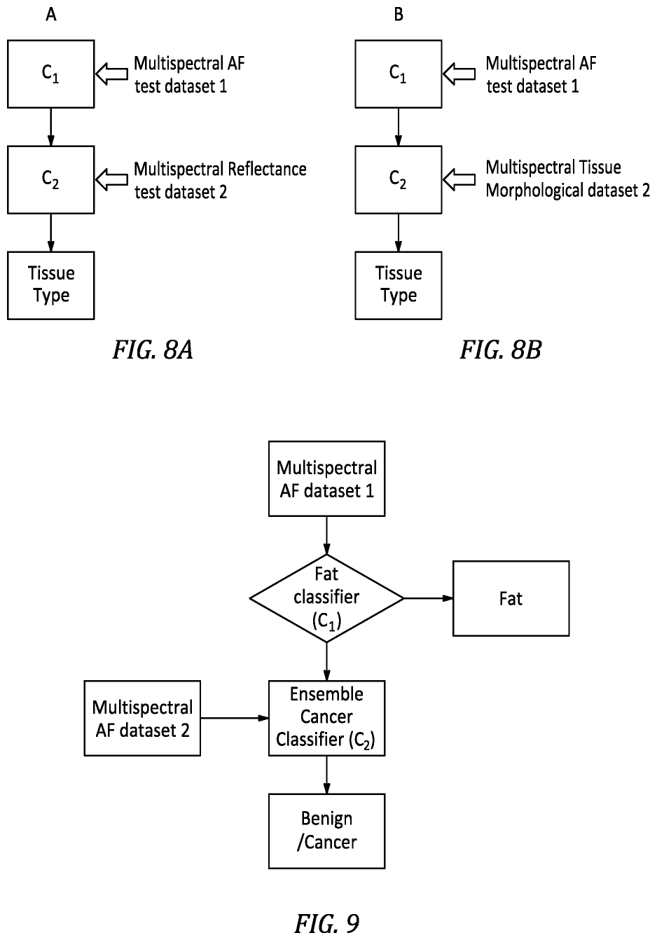

In some present disclosure embodiments, an integrated classifier that combines different classifiers in a cascaded manner may be used; e.g., see FIGS. 8A and 8B. Such an integrated classifier may use different combinations of spectral and imaging data sets such as AF and reflectance data sets (e.g., see FIG. 8A), and AF and morphological attributes (e.g., see FIG. 8B). For instance, a first classifier (i.e., “classifier1” or “C1”) may be used that provides a coarse classification that may yield a higher number of false-positives, and portions of the predicted tissue may then subsequently be analyzed by one or more “computationally more expensive” second classifiers (“classifier2” or “C2”). This approach provides a better classification outcome (e.g., fewer false positives), and also one that reduces overall computation time; i.e., “computationally less expensive”.

In some present disclosure embodiments, a hybrid classifier utilized may have a multi-stage classification architecture, depending on tissue types under analysis. This multi-stage classification architecture (which may be referred to as a “sequential-based ensemble tissue classifier”) may be based on a plurality of data sets including AF multispectral data sets, with an initial “anomaly-based” classifier used to detect and reject one tissue type prior to subsequent meta classification with a second data set (e.g., see FIG. 9). One or more tissue types may be identifiable by a single or series of classification stages such that the aforesaid tissue type(s) may be excluded from further classification with extremely high specificity (i.e., very low rate of false positives).

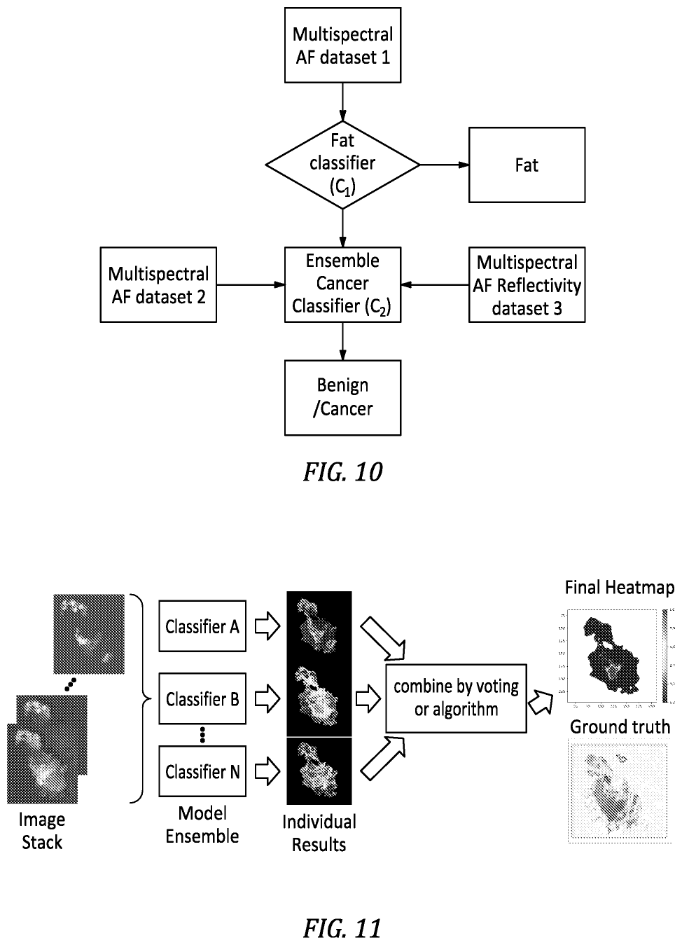

As an example, in breast cancer analysis, tissue can be broadly classified as fat (adipose), benign, or cancerous. Here, the first classifier (C1) may be employed to discern whether the test tissue is fat. In a subsequent step, another classifier (e.g., C2) that is tuned, for example, to differentiate between benign and malignant tissues may be used on non-fat classified tissue for cancer detection. This serial format of “stacked” classifiers allows the stages of classification to be tuned to differentiate the type of tissue at each stage and may enable computationally efficiency (i.e., by elimination of tissue types) and improved analysis by virtue of the “lesser field” of tissue types to be considered. In some embodiments, the present classifier approach may use a plurality of data sets including AF multispectral data sets, with an initial “anomaly-based” classifier to detect and reject one tissue type prior to subsequent meta classification with a plurality of additional multispectral data sets; e.g., see FIG. 10.

In some embodiments, the present disclosure may combine AF imaging with reflectance imaging to compliment and increase the biomolecular and morphological information content and thereby increase the diagnostic power. As stated above, in this manner the present disclosure may leverage the fact that different tissue types absorb different amounts of light at specific wavelengths and therefore the combination of AF and reflectance imaging offers higher diagnostic power.

FIG. 11 shows the application of the present disclosure as is may be used to analyze human breast tissue; e.g., using multiple classifiers on different sets of AF data. Each classifier results in cancer probability, but with different sensitivities and specificity of cancer detection. The output image maps illustrate cancer probability at pixel or superpixel levels. As shown in FIG. 11, while individual classifiers may individually have higher false positives rates, the classifier ensemble approach of the present disclosure provides a more accurate representation when compared with the data acquired from the histopathological images.

While the principles of the disclosure have been described above in connection with specific apparatuses and methods, it is to be clearly understood that this description is made only by way of example and not as limitation on the scope of the disclosure. Specific details are given in the above description to provide a thorough understanding of the embodiments. However, it is understood that the embodiments may be practiced without these specific details.

It is noted that the embodiments may be described as a process which is depicted as a flowchart, a flow diagram, a block diagram, etc. Although any one of these structures may describe the operations as a sequential process, many of the operations can be performed in parallel or concurrently. In addition, the order of the operations may be rearranged. A process may correspond to a method, a function, a procedure, a subroutine, a subprogram, etc.

The singular forms “a,” “an,” and “the” refer to one or more than one, unless the context clearly dictates otherwise. For example, the term “comprising a specimen” includes single or plural specimens and is considered equivalent to the phrase “comprising at least one specimen.” The term “or” refers to a single element of stated alternative elements or a combination of two or more elements unless the context clearly indicates otherwise. As used herein, “comprises” means “includes.” Thus, “comprising A or B,” means “including A or B, or A and B,” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprise”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements may be described as individual units and shown as independent of one another to facilitate the description. In alternative embodiments, such elements may be configured as combined elements.

### REFERENCES

1. Nguyen and Tsien, “Fluorescence-guided surgery with live molecular navigation—a new cutting edge”, Nat Rev Cancer, 13(9), pp. 653-662, 2013.
2. Tummers, et al., “Real-time intraoperative detection of breast cancer using near-infrared fluorescence imaging and methylene blue”, Eur J Surg Oncol., 40(7), 850-858, 2014.
3. Dahr et al., “A diffuse reflectance spectral imaging system for tumor margin assessment using custom annular photodiode arrays”, Biomedical Optics Express, 3, (12), 2012.
4. Pence, I., et al, “Clinical instrumentation and applications of Raman spectroscopy”, Chem Soc Rev.; 45 (7): 1958-1979, 2016.
5. Talari, A. et al., “Raman Spectroscopy of Biological Tissues”, Applied Spectroscopy Reviews, 50:1, 46-111, 2015.
6. Yaroslavsky, A, et al., “Delineating nonmelanoma skin cancer margins using terahertz and optical imaging”, J of Biomedical Photonics & Eng., 3(1), 2017.
7. R. R. Zhang et al., “Beyond the margins: real-time detection of cancer using targeted fluorophores”, Nat Rev Clin Oncol 14, 347-364; 2017
8. Schwarz, R. et al. “Autofluorescence and diffuse reflectance spectroscopy of oral epithelial tissue using a depth-sensitive fiber-optic probe”, Applied Optics, 2008, Vol. 47(6), pp. 825-834.
9. Volynskaya, Z. et al, “Diagnosing breast cancer using diffuse reflectance spectroscopy and intrinsic fluorescence spectroscopy”, Feld. Journal of Biomedical Optics, 2008, Vol. 13(2).
10. Valdez, T. et al, “Multiwavelength Fluorescence Otoscope for Video-Rate Chemical Imaging of Middle Ear Pathology”, Analytical Chemistry, 2014, Vol. 86(20), pp. 10454-10460. Chagnot, C. et al., “Deep UV excited muscle cell autofluorescence varies with the fibre type”, The Analyst, 2015, Vol. 140(12), pp. 4189-4196.
11. Valdez, T. A. et al., “Multi-color reflectance imaging of middle ear pathology in vivo”, Analytical and Bioanalytical Chemistry 2015, Vol. 407(12), pp. 3277-3283.
12. Kho, E. et al., “Broadband hyperspectral imaging for breast tumor detection using spectral and spatial information”, Biomed. Opt. Express 10, 4496-4515 (2019).

1. A method of analyzing an ex-vivo tissue sample, comprising:
sequentially interrogating the tissue sample a plurality of times, each sequential interrogation using at least one excitation light within a plurality of excitation lights and each said excitation light within the plurality of excitation lights centered on a respective wavelength distinct from the respective centered wavelengths of the other excitation lights, wherein at least one of the excitation light centered wavelengths is configured to produce autofluorescence (AF) emissions from one or more biomolecules associated with the tissue sample, and at least one of the excitation light centered wavelengths is configured to produce diffuse reflectance signals from the tissue sample;
using at least one photodetector to detect the AF emissions, or the diffuse reflectance signals, or both from the tissue sample, and to produce photodetector signals representative of the detected said AF emissions, or the detected said diffuse reflectance signals, or both;
processing the photodetector signals attributable to the AF emissions using at least one first classifier trained with a plurality of predetermined AF data sets to determine one or more first data sets indicative of biomolecules present within the tissue sample;
processing the photodetector signals attributable to the diffuse reflectance signals using at least one second classifier trained with a plurality of predetermined diffuse reflectance signal data sets to determine one or more second data sets; and
determining a type of the tissue sample using the one or more first data sets and the one or more second data sets.

2. The method of claim 1, wherein the photodetector signals attributable to the diffuse reflectance signals provide microstructural information relating to the tissue sample.

3. The method of claim 2, wherein the photodetector signals attributable to the diffuse reflectance signals provide morphological information relating to the tissue sample.

4. The method of claim 3, wherein the plurality of predetermined diffuse reflectance signal data sets used to train the second classifier include data sets attributable to known tissue types including benign tissue, fibrous tissue, adipose tissue, diseased tissue, and tissue morphologies.

5. The method of claim 1, wherein the plurality of predetermined AF data sets used to train the first classifier include data sets attributable to known biomolecules.

6. The method of claim 1, wherein the known biomolecules include at least one of tryptophan, collagen, NADH, FAD, elastin, or hemoglobin.

7. The method of claim 1, wherein the at least one first classifier includes a plurality of first classifiers and each producing a said first data set; and
the step of processing the photodetector signals attributable to the AF emissions further includes providing the plurality of first data sets to a first metaclassifier, and the step of determining the type of the tissue sample utilizes an output of the first metaclassifier.

8. The method of claim 1, wherein the at least one first classifier includes a plurality of first classifiers and each producing a said first data set, and the at least one second classifier includes a plurality of second classifiers and each producing a said second data set; and
the step of processing the photodetector signals attributable to the AF emissions further includes providing the plurality of first data sets to a first metaclassifier, and the step of processing the photodetector signals attributable to the diffuse reflectance signals includes providing the plurality of second data sets to the first metaclassifier; and
the step of determining the type of the tissue sample utilizes an output of the first metaclassifier.

9. The method of claim 1, further comprising processing the photodetector signals attributable to the diffuse reflectance signals using at least one third classifier trained with a plurality of predetermined morphology signal data sets to determine one or more third data sets indicative of morphologies present within the tissue sample; and
the step of determining the type of the tissue sample further uses the one or more third data sets indicative of morphologies present within the tissue sample.

10. The method of claim 9, wherein the step of processing the photodetector signals attributable to the AF emissions using at least one first classifier further includes providing the plurality of first data sets to a first metaclassifier, the step of processing the photodetector signals attributable to the diffuse reflectance signals using at least one second classifier includes providing the plurality of second data sets to the first metaclassifier, and the step of processing the photodetector signals attributable to the diffuse reflectance signals using at least one third classifier trained with a plurality of predetermined morphology signal data sets includes providing the plurality of third data sets to the first metaclassifier; and
the step of determining the type of the tissue sample utilizes an output of the first metaclassifier.

11. The method of claim 1, wherein the step of determining the type of the tissue sample utilizes a third classifier that utilizes the one or more first data sets from the at least one first classifier and the one or more second data sets from the at least one second classifier in a cascading manner.

12. The method of claim 1, wherein the step of processing the photodetector signals attributable to the AF emissions and the step of processing the photodetector signals attributable to the diffuse reflectance signals further includes providing a first output from the at least one first classifier and a second output from the at least one second classifier to a second level classifier in an ensemble classifier architecture; and
the step of determining the type of the tissue sample utilizes a third output from the second level classifier.

13. A system for analyzing an ex-vivo tissue sample, comprising:
an excitation light source configured to selectively produce a plurality of excitation lights, each said excitation light centered on a wavelength distinct from the centered wavelength of the other said excitation lights, wherein at least one of the excitation light centered wavelengths is configured to produce autofluorescence (AF) emissions from one or more biomolecules associated with a bladder wall tissue, and diffuse reflectance signals from the tissue sample, the system configured so that the plurality of excitation lights are incident to the tissue sample;
at least one photodetector configured to detect the AF emissions, or the diffuse reflectance signals, or both from the tissue sample as a result of the respective incident excitation light, and produce signals representative of the detected said AF emissions, or the detected said diffuse reflectance signals, or both; and
a system controller in communication with the excitation light source, the at least one photodetector, and a non-transitory memory storing instructions, which instructions when executed cause the system controller to:
control the excitation light unit to sequentially produce the plurality of excitation lights;
control the at least one photodetector to detect the AF emissions, or the diffuse reflectance signals, or both from the tissue sample, and to produce photodetector signals representative of the detected said AF emissions, or the detected said diffuse reflectance signals, or both;
process the photodetector signals attributable to the AF emissions using at least one first classifier trained with a plurality of predetermined AF data sets to determine one or more first data sets indicative of biomolecules present within the tissue sample;
process the photodetector signals attributable to the diffuse reflectance signals using at least one second classifier trained with a plurality of predetermined diffuse reflectance signal data sets to determine one or more second data sets; and
determine a type of the tissue sample using the one or more first data sets and the one or more second data sets.

14. The system of claim 13, wherein the photodetector signals attributable to the diffuse reflectance signals provide microstructural information relating to the tissue sample.

15. The system of claim 14, wherein the photodetector signals attributable to the diffuse reflectance signals provide morphological information relating to the tissue sample.

16. The system of claim 15, wherein the plurality of predetermined diffuse reflectance signal data sets used to train the second classifier include data sets attributable to known tissue types including benign tissue, fibrous tissue, adipose tissue, diseased tissue, and tissue morphologies.

17. The system of claim 13, wherein the plurality of predetermined AF data sets used to train the first classifier include data sets attributable to known biomolecules.

18. The system of claim 13, wherein the known biomolecules include at least one of tryptophan, collagen, NADH, FAD, elastin, or hemoglobin.

19. The system of claim 13, wherein the at least one first classifier includes a plurality of first classifiers and each producing a said first data set; and
wherein the instructions that when executed cause the system controller to process the photodetector signals attributable to the AF emissions, further cause the system controller to provide the plurality of first data sets to a first metaclassifier, and the determination of the tissue sample type utilizes an output of the first metaclassifier.

20. The system of claim 13, wherein the at least one first classifier includes a plurality of first classifiers and each producing a said first data set, and the at least one second classifier includes a plurality of second classifiers and each producing a said second data set; and
wherein the instructions that when executed cause the system controller to process the photodetector signals attributable to the AF emissions, further cause the system controller to provide the plurality of first data sets to a first metaclassifier; and
wherein the instructions that when executed cause the system controller to process the photodetector signals attributable to the diffuse reflectance signals, further cause the system controller to provide the plurality of second data sets to the first metaclassifier; and
wherein the instructions that when executed cause the system controller to determine the type of the tissue sample utilizes an output of the first metaclassifier.

21. The system of claim 13, wherein the instructions that when executed further cause the system controller to process the photodetector signals attributable to the diffuse reflectance signals using at least one third classifier trained with a plurality of predetermined morphology signal data sets to determine one or more third data sets indicative of morphologies present within the tissue sample; and
wherein the instructions that when executed cause the system controller to determine the type of the tissue sample further uses the one or more third data sets indicative of morphologies present within the tissue sample.

22. The system of claim 21, wherein the instructions that when executed cause the system controller to process the photodetector signals attributable to the AF emissions using the at least one first classifier further cause the system controller to provide the plurality of first data sets to a first metaclassifier; and
wherein the instructions that when executed cause the system controller to process the photodetector signals attributable to the diffuse reflectance signals using the at least one second classifier further cause the system controller to provide the plurality of second data sets to the first metaclassifier; and
wherein the instructions that when executed cause the system controller to process the photodetector signals attributable to the diffuse reflectance signals using the at least one third classifier trained with a plurality of predetermined morphology signal data sets further cause the system controller to provide the plurality of third data sets to the first metaclassifier; and
wherein the instructions that when executed cause the system controller to determine the type of the tissue sample uses an output of the first metaclassifier.

23. The system of claim 13, wherein the instructions that when executed cause the system controller to determine the type of the tissue sample utilizes a third classifier that utilizes the one or more first data sets from the at least one first classifier and the one or more second data sets from the at least one second classifier in a cascading manner.

24. The system of claim 13, wherein the instructions that when executed cause the system controller to process the photodetector signals attributable to the AF emissions and to process the photodetector signals attributable to the diffuse reflectance signals further cause the system to provide a first output from the at least one first classifier and a second output from the at least one second classifier to a second level classifier in an ensemble classifier architecture; and
wherein the instructions that when executed cause the system controller to determine the type of the tissue sample utilizes a third output from the second level classifier.
